(19) United States
(12) Patent Application Publication
(10) Pub. No.: US 2008/0279801 A1
(43) Pub. Date: Nov. 13, 2008

(54) Water-In-Oil Microemulsions For Hair Treatment

(76) Inventors: Kelvin Brian Dickinson, Wirral (GB); Anand Ramchandra Mahadeshwar, Hamburg (DE); Ruby Loo Bick Tan-Walker, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/794,021

(22) PCT Filed: Nov. 29, 2005
(86) PCT No.: PCT/EP2005/012907

§ 371 Date: Feb. 25, 2008

(30) Foreign Application Priority Data
Dec. 23, 2004 (EP) 04258090.2

(51) Int. Cl. A61K 8/06; A61Q 5/00

(52) U.S. Cl. 424/70.1

(57) ABSTRACT

The present invention provides a water-in-oil microemulsion for hair treatment comprising: (a) an oil phase comprising: (i) a first oily component which is one or more glyceride fatty esters, and (ii) a second oily component which is one or more hydrocarbon oils of average carbon chain length less than 20 carbon atoms, and (b) a hydrophilic phase comprising: (i) water, (ii) a nonionic emulsifier which is an ethoxylated alcohol having an HLB of at least 6, and (iii) preferably, a hair styling agent.

### FIELD OF THE INVENTION

This invention relates to water-in-oil microemulsions for hair treatment which have enhanced sensory properties and enhanced compatibility with hair benefit agents.

### BACKGROUND OF INVENTION AND PRIOR ART

Consumers oil hair both pre wash and post wash. Pre wash oiling is done as it is believed that oils nourish hair and protect it during the wash process. Post wash oiling is done for manageability and styling. The oiling habit is widely practised by around 800 million people across the Central Asia and Middle East region.

Coconut oil is by far the most common oil used in the Central Asia and Middle East region for hair care. It offers a high level of conditioning benefits, but with the drawback of greasy feel.

EP 1289479 discloses hair oils which incorporate a specific blend of oil types (glyceride fatty esters and hydrocarbon oils) and which can deliver an equivalent level of conditioning benefits to coconut oil, but with superior sensory properties, in particular less greasy feel.

It would be desirable to incorporate hair benefit agents such as hair styling agents into such oils, in order to improve the manageability and styling behaviour of the hair after application of the product.

However a problem is that such agents are generally not compatible with the oil and cannot be incorporated into the oil in a stable manner. When such agents are combined with hair oils at effective levels, they tend to form a two-phase system, with an unattractive appearance and a tendency to separate due to differing density of the two phases.

The present inventors have found that this problem can be solved if a particular type of nonionic emulsifier is formulated with the oil. The invention provides an oil microstructure which has enhanced sensory properties and enhanced compatibility with hair benefit agents such as hair styling agents.

### DEFINITION OF THE INVENTION

The present invention provides a water-in-oil microemulsion for hair treatment comprising:

(a) an oil phase comprising:
(i) a first oily component which is one or more glyceride fatty esters, and
(ii) a second oily component which is one or more hydrocarbon oils of average carbon chain length less than 20 carbon atoms, and
   (b) a hydrophilic phase comprising:
(i) water,
(ii) a nonionic emulsifier which is an ethoxylated alcohol having an HLB of at least 6, and
(iii) preferably, a hair styling agent.

### DETAILED DESCRIPTION OF THE INVENTION

### Microemulsion

By “microemulsion” is meant a thermodynamically or kinetically stable liquid dispersion of an oil phase and a hydrophilic phase. The dispersed phase typically comprises small particles or droplets, with a size range of 5 nm to 200 nm, giving rise to a microemulsion that is transparent or translucent in appearance. This is in contrast to regular (macro-) emulsions that are turbid. The droplets or particles of the microemulsion may be spherical, although other structures are possible. The microemulsion is formed readily and sometimes spontaneously, generally without high-energy input.

### (a) (i) Glyceride Fatty Ester

The water-in-oil microemulsion of the invention comprises an oil phase comprising a first oily component, which is one or more glyceride fatty esters.

By “glyceride fatty esters” is meant the mono-, di-, and trimesters formed between glycerol and long chain carboxylic acids such as C₆-C₃₀ carboxylic acids. The carboxylic acids may be saturated or unsaturated or contain hydrophilic groups such as hydroxyl.

Preferred glyceride fatty esters are derived from carboxylic acids of carbon chain length ranging from C₆ to C₂₄, preferably C₁₀ to C₂₂, most preferably C₁₂ to C₁₈.

Suitable glyceride fatty esters for use in microemulsions of the invention will generally have a viscosity at ambient temperature (25 to 30° C.) of from 0.01 to 0.8 Pa·s, preferably from 0.015 to 0.6 Pa·s, more preferably from 0.02 to 0.065 Pa·s as measured by a Carri-Med CSL2 100 controlled stress rheometer, from TA Instruments Inc., New Castle, Del. (USA).

A variety of these types of materials are present in vegetable and animal fats and oils, such as camellia oil, coconut oil, castor oil, safflower oil, sunflower oil, peanut oil, cottonseed oil, corn oil, olive oil, cod liver oil, almond oil, avocado oil, palm oil, sesame oil, lanolin and soybean oil. These have various ranges of carbon chain lengths depending on the source, typically between about 12 to about 18 carbon atoms. Synthetic oils include trimyristin, triolein and tristearin glyceryl dilaurate. Vegetable derived glyceride fatty esters are particularly preferred, and specific examples of preferred materials for inclusion in microemulsions of the invention as sources of glyceride fatty esters include almond oil, castor oil, coconut oil, sesame oil, sunflower oil and soybean oil. Coconut oil, sunflower oil, almond oil and mixtures thereof are particularly preferred.

The glyceride fatty ester may be present in microemulsions of the invention as a single material or as a blend.

The total content of glyceride fatty ester in microemulsions of the invention suitably ranges from 10% to 95%, preferably from 20% to 80%, by weight based on total weight of the microemulsion.

(a)(ii) Hydrocarbon Oil

The oil phase of the water-in-oil microemulsion of the invention comprises a second oily component, which is one or more hydrocarbon oils of average carbon chain length less than 20 carbon atoms.

Suitable hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will typically contain from about 6 to about 16 carbon atoms, preferably from about 8 up to about 14 carbon atoms. Branched chain hydrocarbon oils can and typically may contain higher numbers of carbon atoms, e.g. from about 6 up to about 20 carbon atoms, preferably from about 8 up to about 18 carbon atoms.

Suitable hydrocarbon oils will generally have a viscosity at ambient temperature (25 to 30° C.) of from 0.0001 to 0.5 Pa·s, preferably from 0.001 to 0.05 Pa·s, more preferably from 0.001 to 0.02 Pa·s as measured by a Carri-Med CSL2 100 controlled stress rheometer, from TA Instruments Inc., New Castle, Del. (USA).

A preferred hydrocarbon oil is light mineral oil. Mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons, in which the number of carbon atoms per hydrocarbon molecule generally ranges from C₁₀ to C₄₀. Mineral oil may be characterised in terms of its viscosity, where light mineral oil is relatively less viscous than heavy mineral oil, and these terms are defined more specifically in the U.S. Pharmacopoeia, 22nd revision, p. 899 (1990). A commercially available example of a suitable light mineral oil for use in the invention is Sirius M40 (carbon chain length C₁₀-C₂₈, mainly C₁₂-C₂₀, viscosity 4.3×10⁻³ Pa·s), available from Silkolene.

Other hydrocarbon oils that may be used in the invention include relatively lower molecular weight hydrocarbons including linear saturated hydrocarbons such a tetradecane, hexadecane, and octadecane, cyclic hydrocarbons such as dioctylcyclohexane (e.g. CETIOL S from Henkel), branched chain hydrocarbons (e.g. ISOPAR L and ISOPAR V from Exxon Corp.).

The hydrocarbon oil may be present in microemulsions of the invention as a single material or as a blend.

The total content of hydrocarbon oil in microemulsions of the invention suitably ranges from 5% to 90%, preferably from 20% to 80%, by weight based on total weight of the microemulsion.

The glyceride fatty ester:hydrocarbon oil weight ratio in microemulsions of the invention may suitably range from 90:10 to 10:90, preferably from 80:20 to 20:80, more preferably from 60:40 to 40:60. Particularly preferred are blends of [coconut oil and/or sunflower oil and/or almond oil] and light mineral oil, in which the [coconut oil and/or sunflower oil and/or almond oil]:light mineral oil weight ratio is about 50:50.

(b)(i) Water

The hydrophilic phase of the water-in-oil microemulsion of the invention comprises water, suitably at a level of from about 2% by weight based on total weight of the microemulsion. Suitably the water level does not exceed about 10% by weight based on total weight of the microemulsion, since this may lead to a hazy product appearance which is undesirable to consumers of hair oils. Preferably the water level ranges from 3 to 7%, more preferably from 4 to 6% by weight based on total weight of the microemulsion.

(b)(ii) Nonionic Emulsifier

The water-in-oil microemulsion of the invention comprises a nonionic emulsifier which is an ethoxylated alcohol having an HLB of at least 6.

Suitable ethoxylated alcohols are commercially available and include the primary aliphatic alcohol ethoxylates and secondary aliphatic alcohol ethoxylates. The length of the polyethenoxy chain can be adjusted to achieve the desired balance between the hydrophobic and hydrophilic elements.

The HLB value of the ethoxylated alcohol suitably ranges from 6 to 12, preferably from 7 to 10, more preferably from 7 to 9.

Examples of suitable ethoxylated alcohols include the condensation products of a higher alcohol (e.g., an alkanol containing about 8 to 16 carbon atoms in a straight or branched chain configuration) condensed with about 2.5 to 20 moles of ethylene oxide.

A preferred group of the foregoing ethoxylated alcohols are the Neodol ethoxylates (Shell Co.), which are higher aliphatic, primary alcohols containing about 9 to 15 carbon atoms condensed with about 2.5 to 20 moles of ethylene oxide. Specific examples are C9 to 11 alkanol condensed with 2.5 to 10 moles of ethylene oxide (Neodol 91-8 or Neodol 91-5), C12 to 13 alkanol condensed with 3 moles ethylene oxide (Neodol 23-3), C12 to 15 alkanol condensed with 12 moles ethylene oxide (Neodol 25-12), C14 to 15 alkanol condensed with 13 moles ethylene oxide (Neodol 45-13), and the like. Such ethoxylates have an HLB (hydrophobic lipophilic balance) value of about 7 to 10. Most preferred is Neodol 23-3, with an HLB of about 8.

The level of nonionic emulsifier in microemulsions of the invention suitably ranges from 10 to 40%, preferably from 15 to 35%, by weight based on total weight of the microemulsion.

(b)(iii) Hair Styling Agent

The hydrophilic phase of the water-in-oil microemulsion of the invention preferably comprises a hair styling agent.

The hair styling agent can be a hair fixative or film former that imparts style-retention properties to hair, i.e., sets the hair.

Hair fixatives and film formers are typically polymeric in nature and many such polymers are available commercially which contain groups which render the polymers cationic, anionic, amphoteric or nonionic in nature (hereinafter referred to as “hair styling polymers”).

Examples of suitable anionic hair styling polymers are:

copolymers of vinyl acetate and crotonic acid; terpolymers of vinyl acetate, crotonic acid and a vinyl ester of an alpha-branched saturated aliphatic monocarboxylic acid such as vinyl neodecanoate; copolymers of methyl vinyl ether and maleic anhydride (molar ratio about 1:1) wherein such copolymers are 50% esterified with a saturated alcohol containing from 1 to 4 carbon atoms such as ethanol or butanol; acrylic copolymers containing acrylic acid or methacrylic acid as the anionic radical-containing moiety with other monomers such as: esters of acrylic or methacrylic acid with one or more saturated alcohols having from 1 to 22 carbon atoms (such as methyl methacrylate, ethyl acrylate, ethyl methacrylate, n-butyl acrylate, t-butyl acrylate, t-butyl methacrylate, n-butyl methacrylate, n-hexyl acrylate, n-octyl acrylate, lauryl methacrylate and behenyl acrylate); glycols having from 1 to 6 carbon atoms (such as hydroxypropyl methacrylate and hydroxyethyl acrylate); styrene; vinyl caprolactam; vinyl acetate; acrylamide; alkyl acrylamides and methacrylamides having 1 to 8 carbon atoms in the alkyl group (such as methacrylamide, t-butyl acrylamide and n-octyl acrylamide); and other compatible unsaturated monomers.

Specific examples of suitable anionic hair styling polymers are:

RESYN® 28-2930 available from National Starch (vinyl acetate/crotonic acid/vinyl neodecanoate copolymer); Ultrahold® 8 available from BASF (CTFA designation Acrylates/acrylamide copolymer); the Gantrez® ES series available from ISP corporation (esterified copolymers of methyl vinyl ether and maleic anhydride).

Other suitable anionic polymers include hydrophilically-modified polyurethanes. Examples of such materials are carboxylated polyurethanes, which are linear, hydroxyl-terminated copolymers having pendant carboxyl groups. They may be ethoxylated and/or propoxylated at least at one terminal end. The carboxyl group can be a carboxylic acid group or an ester group, wherein the alkyl moiety of the ester group contains one to three carbon atoms. The carboxylated polyurethane resin can also be a copolymer of polyvinylpyrrolidone and a polyurethane, having a CTFA designation PVP/polycarbamyl polyglycol ester. Suitable carboxylated polyurethane resins are disclosed in EP 0 619 111 A1 and U.S. Pat. No. 5,000,955. Other suitable hydrophilic polyurethanes are disclosed in U.S. Pat. Nos. 3,822,238; 4,156,066; 4,156,067; 4,255,550; and 4,743,673.

Good results have been obtained with carbamate group modified polyurethanes comprising units having the formula:

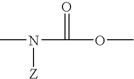

wherein Z is hydrogen, an alkyl, fully or partially fluorinated alkyl or acyl group, provided that not all of the Z groups in the polyurethane are hydrogen. These materials are described in U.S. Pat. No. 6,730,289.

Examples of suitable amphoteric hair styling polymers are those containing cationic groups derived from monomers such as t-butyl aminoethyl methacrylate as well as carboxyl groups derived from monomers such as acrylic acid or methacrylic acid. One specific example of an amphoteric polymer is AMPHOMER (Octylacrylamide/acrylates/butylaminoethyl methacrylate copolymer) sold by the National Starch and Chemical Corporation.

Examples of suitable nonionic hair styling polymers are homopolymers of N-vinylpyrrolidone and copolymers of N-vinylpyrrolidone with compatible nonionic monomers such as vinyl acetate. Nonionic polymers containing N-vinylpyrrolidone in various weight average molecular weights are available commercially from ISP Corporation. Specific examples of such materials are homopolymers of N-vinylpyrrolidone having an average molecular weight of about 630,000 sold under the name PVP K-90 and are homopolymers of N-vinylpyrrolidone having an average molecular weight of about 1,000,000 sold under the name of PVP K-120.

Other suitable nonionic hair styling polymers are cross-linked silicone resins or gums. Specific examples include rigid silicone polymers such as those described in EP-A-240 350 and cross-linked silicone gums such as those described in WO 96/31188.

Examples of suitable cationic hair styling polymers are copolymers of amino-functional acrylate monomers such as lower alkyl aminoalkyl acrylate, or methacrylate monomers such as dimethylaminoethyl methacrylate, with compatible monomers such as N-vinylpyrrolidone, vinyl caprolactam, alkyl methacrylates (such as methyl methacrylate and ethyl methacrylate) and alkyl acrylates (such as ethyl acrylate and n-butyl acrylate).

Specific examples of suitable cationic hair styling polymers are:

copolymers of N-vinylpyrrolidone and dimethylaminoethyl methacrylate, available from ISP Corporation as Copolymer 845, Copolymer 937 and Copolymer 958;
copolymers of vinylpyrrolidone and dimethylaminopropylacrylamide or methacrylamide, available from ISP Corporation as Styleze CC10;
Polyquaternium-11 (formed by the reaction of diethyl sulphate and a copolymer of vinyl pyrrolidone and dimethyl aminoethylmethacrylate), available from ISP as Gafquat® 734, 755 and 755N, and from BASF as Luviquat® PQ11;
Polyquaternium-16 (formed from methylvinylimidazolium chloride and vinylpyrrolidone), available from BASF as Luviquat® FC 370, FC 550, FC 905 and HM-552;
Polyquaternium-46 (prepared by the reaction of vinylcaprolactam and vinylpyrrolidone with methylvinylimidazolium methosulphate), available from BASF as Luviquat®Hold.

With certain of the above-described hair styling polymers it may be necessary to neutralise some acidic groups to promote solubility/dispersibility. Examples of suitable neutralising agents include 2-amino-2-methyl-1,3-propanediol (AMPD); 2-amino-2-ethyl-1,3-propanediol (AEPD); 2-amino-2-methyl-1-propanol (AMP); 2-amino-1-butanol (AB); monoethanolamine (MEA); diethanolamine (DEA); triethanolamine (TEA); monoisopropanolamine (MIPA); diisopropanol-amine (DIPA); triisopropanolamine (TIPA); and dimethyl stearamine (DMS). A long chain amine neutralising agent such as lauramidopropyl dimethylamine may be employed, as is described in U.S. Pat. No. 4,874,604.

A preferred class of hair styling agent is a solid particulate material, which is able to impart body and stylability to the hair.

Preferred solid particulate materials have a D3,2 average particle size in the range from 10 to 700, preferably from 10 to 500, more preferably from 20 to 300, yet more preferably from 20 to 200, and most preferably from 30 to 150 nm, for example about from 50 to 100 nm.

It is preferred that the solid particulate materials be colloidal in an aqueous dispersion.

The solid particulate material can be a primary particle or an aggregate. Preferably, it is a primary particle.

Suitably, the solid particles are relatively hard and typically have a Youngs Modulus of more than 0.01, preferably more than 0.1, more preferably more than 1.0, yet more preferably more than 4 GPa, and yet more preferably more than 10 GPa.

The solid particulate materials can be organic or inorganic in nature. Furthermore, the solid particulate material may be composed entirely of one material or may consist of a composite of materials.

Examples of suitable solid particulate materials include polymers, which are preferably cross-linked, (e.g. polystyrene, silicone elastomer powders, PTFE, rubber), silicas, alumina, aluminosilicate, clays and colloidal metals (e.g. titanium dioxide, zinc oxide).

A preferred class of solid particulate materials are silicas, such as silica gels, hydrated silicas and precipitated silicas (e.g. Cab-O-Sil and Aerosil, from Cabot Corp. and Degussa respectively).

A particularly preferred class of silicas are the colloidal silicas. Suitable examples include Ludox HS-40, Ludox TM-40, Ludox SM, Ludox CL and Ludox AM (from Grace Davison Products).

Mixtures of any of the above described hair styling agents may also be used.

The total amount of hair styling agent suitably ranges from 0.05 to 0.5%, preferably from 0.1 to 0.3%, by weight based on total weight of the microemulsion.

### Process

Water-in-oil microemulsions according to the present invention form spontaneously and may be prepared by simple mixing at ambient temperature.

A preferred process for preparing a water-in-oil microemulsion according to the present invention comprises the following steps:

(I) forming a solution or dispersion of the styling agent [(b)(iii)] in the water [(b)(i)];
(II) forming a separate mixture of the oil phase [(a)] and nonionic emulsifier [(b)(ii)];
(III) blending the solution or dispersion obtained in (I) with the mixture obtained in (II).

### Product Form and Usage

Compositions of this invention are preferably for application directly to the hair in neat form, either before or after shampooing.

Accordingly the invention also provides a method of treating hair comprising the step of applying a water-in-oil microemulsion as described above directly to the hair as a pre-wash treatment or as a post-wash treatment.

### Optional Ingredients

Compositions of this invention may contain any other ingredient normally used in hair treatment formulations. These other ingredients may include preservatives such as Phenoxetol® (2-phenoxyethanol), colouring agents, antioxidants such as BHT (butylhydroxytoluene), fragrances and antimicrobials such as Glycacil-L® (iodopropynyl butylcarbamate). Each of these ingredients will be present in an amount effective to accomplish its purpose. Generally these optional ingredients are included individually at a level of up to about 5% by weight based on total weight of the microemulsion.

The invention is further illustrated by way of the following Examples, in which all percentages are by weight based on total weight unless otherwise stated.

### EXAMPLES

Water-in-oil microemulsions containing hair styling agents were prepared, having ingredients as shown in the following Table:

| | Formulation Examples: | | |
|---|---|---|---|
| Ingredient | Example 1 | Example 2 | Example 3 |
| Sunflower oil | 32.5 | 32.5 | 32.5 |
| Light mineral oil | 32.5 | 32.5 | 32.5 |
| (Sirius M40, from | | | |
| Silkolene) | | | |
| Nonionic emulsifier | 30.0 | 30.0 | 30.0 |
| (NEODOL 23-3, from | | | |
| Shell Co.) | | | |
| Water | to 100 | to 100 | to 100 |
| Colloidal silica | — | 0.25 (a.i.) | — |
| (LUDOX CL, from | | | |
| Degussa) | | | |
| Colloidal silica | — | — | 0.25 (a.i.) |
| (LUDOX TM-40, from | | | |
| Degussa) | | | |

Comparative evaluations of the above formulations according to the invention were carried out using a control formulation of 50 wt % Sirius M40 and 50 wt % sunflower oil.

The formulations of Examples 1 to 3 were each compared against the control formulation across a number of performance attributes. Evaluation was carried out in two stages:

### (i) Post Oiling.

Half of the hair of a mannequin head was oiled with the control formulation and the other half with the test formulation (Example 1, 2 or 3 respectively). 2.0 ml of formulation was used to oil the individual half head. After one hour the mannequin head was assessed by an expert salon hairdresser.

### (ii) Post Wash.

3.5 ml of a commercial shampoo was measured and applied onto the oiled half head, followed by washing and rinsing in accordance with normal procedures. The shampooing and rinsing procedure was repeated for a second application. The same procedure was followed for the other oiled half head. After washing and rinsing was complete the mannequin head was allowed to dry at normal temperature (20 to 25 degrees C.). On drying the mannequin head was assessed by an expert salon hairdresser.

The following results were obtained:

### Post Oiling:

Compared to the control, the formulation of Example 1 gave significantly (>90%) better hair body. The formulation of Example 1 was also found to have significantly (>95%) reduced product sticky feel and significantly (>90%) better product spread compared to the control.

Compared to the control, the formulation of Example 2 gave significantly (>95%) better hair body and significantly (>99%) reduced hair sticky feel.

Compared to the control, the formulation of Example 3 gave significantly (>95%) better hair body and significantly (>90%) better conditioning.

### Post Wash:

Compared to the control, the formulation of Example 1 gave significantly (>90%) better hair smoothness and significantly (>99%) better hair gloss.

Compared to the control, the formulation of Example 2 gave significantly (>90%) better hair body.

Compared to the control, the formulation of Example 3 gave significantly (>90%) better hair body, significantly (>90%) better hair conditioning and significantly (>90%) better hair shine.

In a further test, the control formulation was blended directly with LUDOX TM-40 (0.25% a.i.).

This resulted in an unstable formulation in which water droplets from the colloidal silica settled at the bottom of the storage jar immediately. By contrast, the formulation of Example 3 remained stable on storage for over 6 months at 25 degrees C.

Also, no significant differences in hair body were observed, either post oiling or post wash, when the control formulation blended with LUDOX TM-40 (0.25% a.i.) was tested against the control formulation per se (i.e. without LUDOX TM-40), according to the evaluation protocol described above.

1. A water-in-oil microemulsion for hair treatment comprising:
(a) an oil phase comprising:
(i) a first oily component which is one or more glyceride fatty esters, and
(ii) a second oily component which is one or more hydrocarbon oils of average carbon chain length less than 20 carbon atoms, and
(b) a hydrophilic phase comprising:
(i) water,
(ii) a nonionic emulsifier which is an ethoxylated alcohol having an HLB of at least 6, and
(iii) preferably, a hair styling agent.

2. A microemulsion according to claim 1, in which the source of glyceride fatty esters is selected from coconut oil, sunflower oil, almond oil and mixtures thereof.

3. A microemulsion according to claim 1, in which the total content of glyceride fatty ester ranges from 20% to 80% by weight based on total weight of the microemulsion.

4. A microemulsion according to claim 1, in which the hydrocarbon oil is light mineral oil.

5. A microemulsion according to claim 1, in which the total content of hydrocarbon oil ranges from 20% to 80% by weight based on total weight of the microemulsion.

6. A microemulsion according claim 1, in which the glyceride fatty ester:hydrocarbon oil weight ratio ranges from 95:5 to 5:95, preferably from 90:10 to 10:90, most preferably from 80:20 to 20:80.

7. A microemulsion according to claim 1, in which the water level ranges from 3 to 7%, more preferably from 4 to 6% by weight based on total weight of the microemulsion.

8. A microemulsion according to claim 1, in which the HLB value of the ethoxylated alcohol ranges from 6 to 12, preferably from 7 to 10, more preferably from 7 to 9.

9. A microemulsion according to claim 8, in which the ethoxylated alcohol is a higher aliphatic, primary alcohol containing about 9 to 15 carbon atoms, condensed with about 2.5 to 10 moles of ethylene oxide.

10. A microemulsion according to claim 9, in which the ethoxylated alcohol is C12 to 13 alkanol condensed with 3 moles ethylene oxide.

11. A microemulsion according to claim 1, which comprises a hair styling agent which is a hair fixative or film former.

12. A microemulsion according to claim 11, in which the hair fixative or film former is a carbamate group modified polyurethane comprising units having the formula: 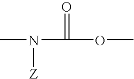
wherein Z is hydrogen, an alkyl, fully or partially fluorinated alkyl or acyl group, provided that not all of the Z groups in the polyurethane are hydrogen.

13. A microemulsion according to claim 1, which comprises a hair styling agent which is a solid particulate material which is able to impart body and stylability to the hair.

14. A microemulsion according to claim 13, in which the solid particulate material is colloidal silica.

15. A method of treating hair comprising the step of applying a water-in-oil microemulsion according claim 1 directly to the hair as a pre-wash treatment or as a post-wash treatment.
